(19) United States
(12) Patent Application Publication
(10) Pub. No.: US 2014/0121610 A1
(43) Pub. Date: May 1, 2014

(54) Garment and Method Used to Provide Hydration and Elasticity to the Skin of Abdomen Area

(71) Applicants: Lelia E. Woodley, Memphis, TN (US); Nicole Crawley, Collierville, TN (US)

(72) Inventors: Lelia E. Woodley, Memphis, TN (US); Nicole Crawley, Collierville, TN (US)

(21) Appl. No.: 13/663,799

(22) Filed: Oct. 30, 2012

(51) Int. Cl. A61M 35/00

(52) U.S. Cl. CPC A61M 35/00
USPC 604/290; 604/304; 604/307; 604/308

(57) ABSTRACT

A garment used to provide hydration and elasticity to the skin of the abdomen area is disclosed. The garment comprises a detachable front panel configured to encase and secure to the abdomen area. The front panel comprises an inner layer and an outer layer each having an interior side and an exterior side. The exterior side of the inner layer is contiguous to the interior side of the outer layer. The inner layer comprises a gelatinous material comprising proteins. In one embodiment, the interior side of the inner layer comprises a self-adhesive edge which secures the front panel to the abdomen area. In another embodiment, the garment comprises a back strap which secures the front panel to the abdomen area at a first and second region. In one embodiment, the front panel may comprise a single layer having an interior side and an exterior side, wherein the interior side may be lined with gelatinous material or may be pretreated with micro particles and/or micro capsules comprising vitamins and ingredients having skin nourishing and skin regenerative qualities. A method used to provide hydration and elasticity to the skin of the abdomen area is also disclosed.

### FIELD

The present invention relates generally to a garment and method used to provide hydration and elasticity to the skin of the abdomen area during pregnancy. More specifically, the present invention relates to a garment and method used to provide hydration and elasticity to the skin of the abdomen area by providing vitamins and ingredients having skin nourishing and skin regenerative qualities to the abdomen area as it gradually expands throughout a women's pregnancy, thereby reducing the likelihood of stretch marks and other undesired effects resulting from such expansion.

### BACKGROUND

During pregnancy, women are highly susceptible to stretch marks on and around the abdomen area as the uterus gradually expands during each of the three trimesters of a woman's pregnancy. Additionally, women are susceptible to uncomfortable itching and dryness that are present as the skin of the abdomen area expands, stretches and tightens during the course of a woman's pregnancy.

Skin care products or topical aids, such as lotions, exist that may provide relief and/or a solution for pregnancy related conditions, including stretch marks, dry skin and itchy skin, that occur as the abdomen area expands over the course of a pregnancy. These topical aids often contain vitamins and other ingredients that maximize the skin's elasticity, promote hydration and skin regeneration, and soothe the skin during the first, second and even third trimesters. Consistent use of such topical aids allows the skin of the abdomen area to withstand the ongoing stretching of the abdomen as the skin stretches and grows. Such skin care products are typically effective for a limited amount of time, such as several hours, and require consistent reapplication to the skin.

Hydration of the skin to promote skin cell regeneration is more effective at night. Research shows that skin cell regeneration almost doubles at night, peaking from about 11:00 pm to 4:00 am boosting the production of collagen. While an individual sleeps, her skin temperature rises allowing skin care products to be better absorbed by the skin, allowing greater results. Water loss is also increased at night while sleeping due to a quantity of water that passes from inside a body through the epidermal layer to the surrounding atmosphere via diffusion and evaporation processes. Such water loss causes the skin to be dry and itchy at night and in the early morning, thus, moisturizing at night improves the skins barrier. Also, nocturnal blood flow is higher in skin at night. Thus, since the blood flow is increased in the skin, the surface area of absorption of the skin also increases at night.

Accordingly, a garment and method used to provide hydration and elasticity to the abdomen area during pregnancy, wherein such garment and method effectively provides hydration for a continuous and substantial period of time is needed. Moreover, a garment and method used to provide hydration and elasticity to the abdomen area during pregnancy wherein the garment and method accommodate the continuous and progressive growth of the abdomen area is needed. In addition, a garment and method used to provide hydration and elasticity to the abdomen area during pregnancy, wherein the garment may be worn at night to provide maximum hydration and elasticity is needed.

### BRIEF SUMMARY

A garment used to provide hydration and elasticity to the skin of the abdomen area is disclosed. In one embodiment, the garment may comprise a detachable front panel configured to encase and secure to the abdomen area. In one embodiment, the front panel comprises an inner layer having an interior side and an exterior side. The front panel may also comprise an outer layer having an interior side and an exterior side. In one embodiment, the exterior side of the inner layer may be contiguous to the interior side of the outer layer. In one embodiment, the inner layer comprises a gelatinous material comprising proteins.

In one embodiment, the interior side of the inner layer of the garment may comprise a self-adhesive edge which secures the front panel to the abdomen area. In another embodiment, the garment may comprise a back strap which secures the front panel to the abdomen area at a first region and a second region. In one embodiment, the back strap may be secured to the front panel by a closure structure. The closure structure may include hook and loop fasteners, snap fasteners, button fasteners, lace fastener, hook fasteners and/or any mixtures thereof.

In one embodiment, the gelatinous material of the inner layer may comprise any of the following ingredients: hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof.

In another example embodiment, a garment used to provide hydration and elasticity to the skin of the abdomen area may comprise a detachable front panel configured to encase and secure to the abdomen area, wherein the front panel comprises a single layer having an interior side and an exterior side. In one embodiment, the interior side of the front panel may be pretreated with micro particles and/or micro capsules comprising vitamins and ingredients having skin nourishing and skin regenerative qualities.

In one embodiment, the interior side of the front panel may comprise a self-adhesive edge which secures the front panel to the abdomen area. In another embodiment, the garment may comprise a back strap which secures the front panel to the abdomen area at a first region and second region. The back strap may be secured to the front panel by a closure structure which may include hook and loop fasteners, snap fasteners, button fasteners, lace fastener, hook fasteners and/or any combinations or mixtures thereof.

In one embodiment, the micro particles and/or micro capsules may comprise the following ingredients: proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof.

A method for providing hydration and elasticity to the skin of the abdomen area is also disclosed. In one embodiment, the method may comprise the following steps: (a) obtaining a garment comprising a detachable front panel configured to encase and secure to the abdomen area, wherein the front panel comprises an inner layer having an interior side and an exterior side and an outer layer having an interior side and an exterior side, wherein the exterior side of the inner layer is contiguous to the interior side of the outer layer, wherein the inner layer comprises a gelatinous material comprising proteins; (b) securing the garment to the abdomen area; and (c) wearing the garment for a period of time. In one embodiment, the method may further include the following steps: step (d) removing the garment and step (e) repeating steps (b) to (c) as needed.

In one embodiment, the interior side of the inner layer may comprise a self-adhesive edge which secures the front panel to the abdomen area. In another embodiment, the garment may comprise a back strap which secures the front panel to the abdomen area at a first region and a second region.

In one embodiment, the gelatinous material may further comprise the following ingredients: hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter, and any mixtures thereof.

Another exemplary method for providing hydration and elasticity to the skin of the abdomen area is disclosed. The method may comprise the following steps: (a) obtaining a garment comprising a detachable front panel configured to encase and secure to the abdomen area, wherein the front panel may comprise a single layer having an interior side and an exterior side, wherein the interior side is pretreated with micro particles and/or micro capsules comprising vitamins and ingredients having skin nourishing and skin regenerative qualities; (b) securing the garment to the abdomen area; and (c) wearing the garment for a period of time. In one embodiment, the method may further include the following steps: step (d) removing the garment and step (e) repeating steps (b) to (c) as needed.

In one embodiment, the interior side of the front panel may comprise a self-adhesive edge which secures the front panel to the abdomen area. In another embodiment, the micro particles and/or micro capsules may comprise the following ingredients: proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof.

### BRIEF DESCRIPTION OF THE DRAWINGS

The present disclosure will be better understood by reference to the following detailed description when considered in conjunction with the accompanying drawing wherein:

FIG. 1 is a isometric perspective view of a front panel of a garment used to provide hydration and elasticity to the skin of the abdomen area in accordance with an exemplary embodiment of the present disclosure.

FIG. 2 is a perspective left side view of the front panel of the garment used to provide hydration and elasticity to the skin of the abdomen area as depicted in FIG. 1.

FIG. 3 is a perspective right side view of the front panel of the garment used to provide hydration and elasticity to the skin of the abdomen area as depicted in FIG. 1.

FIG. 4 is a perspective view of the inner layer of the front panel of the garment used to provide hydration and elasticity to the skin of the abdomen area as depicted in FIG. 1.

FIG. 5 is a isometric perspective view of a front panel of a garment used to provide hydration and elasticity to the skin of the abdomen area in accordance with another exemplary embodiment of the present disclosure.

FIG. 6 is a perspective left side view of the front panel of the garment used to provide hydration and elasticity to the skin of the abdomen area as depicted in FIG. 5.

FIG. 7 is a perspective right side view of the front panel of the garment used to provide hydration and elasticity to the skin of the abdomen area as depicted in FIG. 5.

FIG. 8 is a perspective view of the interior side of the single layer of the front panel of the garment used to provide hydration and elasticity to the skin of the abdomen area as depicted in FIG. 5.

FIG. 9 is a perspective view of the interior side of the inner layer of the front panel as depicted in FIG. 1 wherein the interior side of the inner layer is lined with a self-adhesive edge.

FIG. 10 is a perspective view of the interior side of the single layer front panel as depicted in FIG. 5 wherein the interior side of the single layer is lined with a self-adhesive edge.

FIG. 11 is a perspective view of the front panel of a garment used to provide hydration and elasticity to the skin of the abdomen area, wherein the exterior side of the outer layer has a first region and a second region in accordance with an exemplary embodiment of the present disclosure.

FIG. 12 is a perspective side view of the front panel of a garment used to provide hydration and elasticity to the skin of the abdomen area as depicted in FIG. 11.

FIG. 13 is a perspective view of the lower portion of the front panel of FIG. 11 applied to the lower abdominal area of a user.

FIG. 14 is a perspective view of a back strap of a garment used to provide hydration and elasticity to the skin of the abdomen area in accordance with an exemplary embodiment of the present disclosure. FIG. 14 depicts the interior side of the back strap having first region and a second region.

FIG. 15 is a perspective view of the exterior side of the back strap of the garment used to provide hydration and elasticity to the skin of the abdomen area as shown in FIG. 14.

FIG. 16 is a perspective side view of a garment used to provide hydration and elasticity to the skin of the abdomen area in accordance with an exemplary embodiment of the present disclosure. FIG. 16 depicts the garment secured to its user wherein the back strap is secured to the front panel.

FIG. 17 is a perspective front view of a garment used to provide hydration and elasticity to the skin of the abdomen area secured to its user as shown in FIG. 16.

FIG. 18 is perspective back view of a garment used to provide hydration and elasticity to the skin of the abdomen area secured to its user as shown in FIG. 16.

FIG. 19 is a perspective view of the front panel of a garment used to provide hydration and elasticity to the skin of the abdomen area, wherein the exterior side of the outer layer has a first region and a second region in accordance with a second exemplary embodiment of the present disclosure.

FIG. 20 is a perspective side view of a front panel of a garment used to provide hydration and elasticity to the skin of the abdomen area as depicted in FIG. 19.

FIG. 21 is a perspective view of the front panel of the garment used to provide hydration and elasticity to the skin of the abdomen area as shown in FIG. 19. FIG. 21 depicts the front panel as applied to the lower abdomen area of its user.

FIG. 22 is a perspective view of a back strap of a garment used to provide hydration and elasticity to the skin of the abdomen area in accordance with a second exemplary embodiment of the present disclosure. FIG. 22 depicts the interior side of the back strap having first region and second region.

FIG. 23 is a perspective side view of a garment used to provide hydration and elasticity to the skin of the abdomen area secured to the user in accordance with a further exemplary embodiment of the present disclosure. FIG. 23 depicts the back strap as shown in FIG. 23 secured to the front panel as shown in FIG. 19.

FIG. 24 is a perspective front view of a garment used to provide hydration and elasticity to the skin of the abdomen area as shown in FIG. 23.

FIG. 25 is perspective back view of a garment used to provide hydration and elasticity to the skin of the abdomen area as shown in FIG. 23.

FIG. 26 is a perspective view of a back strap of a garment used to provide hydration and elasticity to the skin of the abdomen area in accordance with another exemplary embodiment of the present disclosure. FIG. 26 depicts the interior side of the back strap having first region and second region.

FIG. 27 is a perspective view of a back strap of a garment used to provide hydration and elasticity to the skin of the abdomen area in accordance with still a further exemplary embodiment of the present disclosure. FIG. 27 depicts the interior side of the back strap having first region and second region.

FIG. 28 is a perspective side view of a front panel of a garment used to provide hydration and elasticity to the skin of the abdomen area in accordance to a further exemplary embodiment, wherein the exterior side of the single layer has a first region.

FIG. 29 is a perspective front view of the front panel as depicted in FIG. 28, wherein the first region and second region of exterior side of the single layer are shown.

FIG. 30 is a perspective side view of a front panel of a garment used to provide hydration and elasticity to the skin of the abdomen area in accordance to still a further exemplary embodiment, wherein the exterior side of the outer layer has a first region.

FIG. 31 is a perspective front view of the front panel as depicted in FIG. 30, wherein the first region and second region of exterior side of the outer layer are shown.

### DETAILED DESCRIPTION

Garment 1 used to provide hydration and elasticity to the skin of the abdomen area is disclosed. In one embodiment, the garment may comprise a detachable front panel 2 configured to encase and secure to the abdomen area. In one embodiment, front panel 2 is configured to cover a substantial portion of the abdomen area of a pregnant woman. In one embodiment, front panel 2 is configured to cover the abdomen area from the top of woman's uterus down to the bottom of women's uterus which is typically above the pubic region. In another embodiment, front panel 2 is configured to cover the sides of the pregnant person's abdomen area at and/or above the hip region. The shape and configuration of front panel 2 may vary as desired by one of skill in the art.

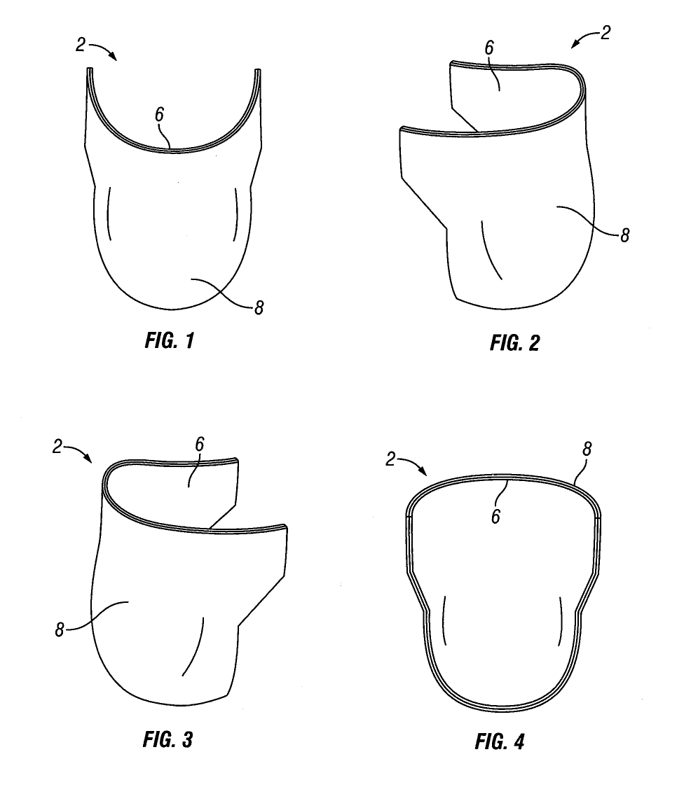

Referring now to FIGS. 1 to 4, in one embodiment, the front panel 2 may comprise an inner layer 6 and an outer layer 8, each having an interior side and an exterior side. In one embodiment, the exterior side of the inner layer 6 may be contiguous to the interior side of the outer layer 8. In one embodiment, the inner layer 6 comprises a gelatinous material comprising proteins and other vitamins and ingredients than contain skin nourishing and skin regenerative qualities to provide hydration to the skin of the abdomen area when front panel 2 is secured to a user. In one embodiment, the gelatinous material of the inner layer may comprise any of the following ingredients: hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof. Gelatinous material may include any other ingredients with skin nourishing and skin regenerative qualities as desired by one skilled in the art.

In one embodiment, outer layer 8 of front panel 2 may be made of any semi stretch, smooth fabric, textile or blend of textiles such as cotton, nylon and/or elastic as desired by one of skill in the art. Any textile or blend of textiles may be used as long as such textile or blend of textiles is capable of stretching (such as four way stretching) and moisture wicking. In one embodiment, outer layer 8 may be made of a textile or blended textile that is preshrunk. In one embodiment, outer layer 8 may comprise silicone or a blend thereof wherein such blend may include, but is not limited to, silicone, polyester and/or foam. Outer layer 8 may be made of other materials or blends thereof as desired by one skilled in the art.

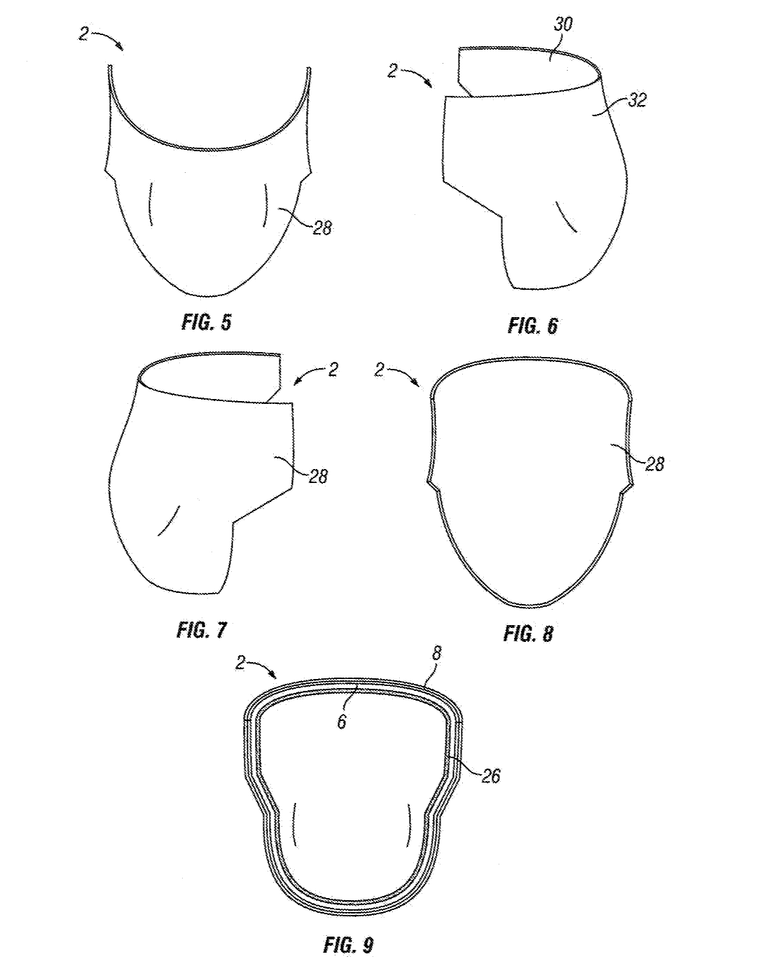

Referring now to FIGS. 5 to 8, in another embodiment, front panel 2 may comprise a single layer 28 having an interior side 30 and an exterior side 32. In one embodiment, single layer 28 may be made of any semi stretch, smooth fabric, textile or blend of textiles such as cotton, nylon and/or elastic as desired by one of skill in the art. Any textile or blend of textiles may be used as long as such textile or blend of textiles is capable of stretching (such as four way stretching) and moisture wicking. In one embodiment, the single layer 28 is made of a textile or blend of textiles that is preshrunk. In one embodiment, single layer 28 may comprise silicone or a blend thereof wherein such blend may include, but is not limited to, silicone, polyester and/or foam. Single layer 28 may be made of other materials or blends thereof as desired by one skilled in the art. In one embodiment, the single layer 28 of front panel 2 may be pretreated with micro particles and/or micro capsules comprising vitamins and ingredients having skin nourishing and skin regenerative qualities to provide hydration to the skin of the abdomen area. In one embodiment, the micro particles and/or micro capsules may comprise, but are not limited to, the following ingredients: proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof.

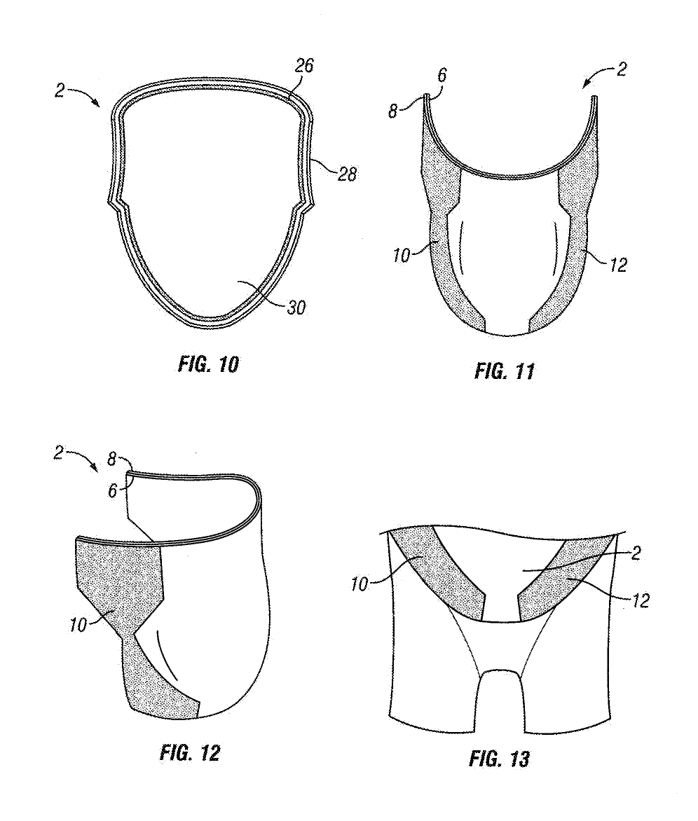

Referring to FIGS. 9 and 10, in one embodiment, front panel 2 of the garment may comprise a self-adhesive edge which secures the front panel 2 to the abdomen area of a user. In one embodiment, self-adhesive edge 26 may be contiguous to the interior side of inner layer 6 as shown in FIG. 9. In another embodiment, self-adhesive edge 26 may be contiguous to the interior side of single layer 28 as shown in FIG. 10. In one embodiment, self-adhesive edge 26 may be continuous around and near the perimeter of front panel 2. In a further embodiment, self-adhesive edge 26 may be non-continuous sections or strips positioned around the entire perimeter or portions of the perimeter of front panel 2, or the self-adhesive edge 26 may be arranged around the perimeter and/or non-perimeter portions of front panel 2. Self-adhesive edge 26 may be positioned or arranged in any configuration, as desired by the skilled artisan, that allows for front panel 2 to be secured to the abdomen area of a user.

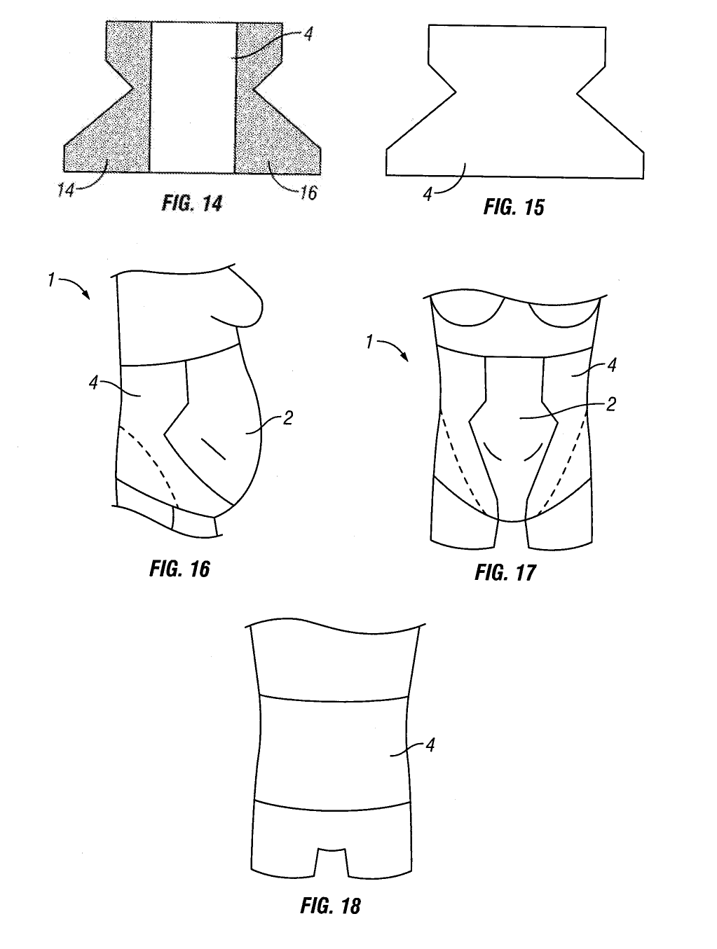

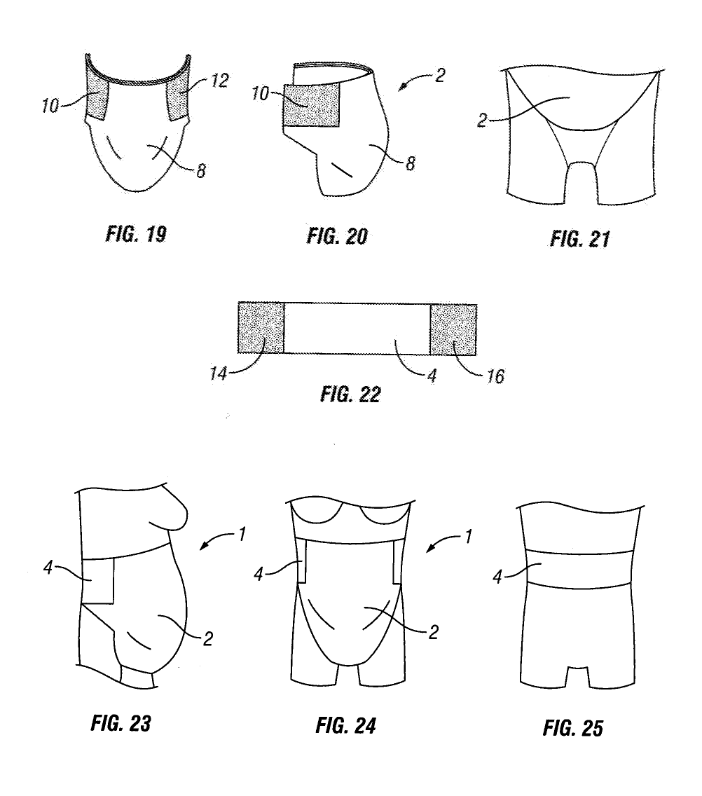

Referring to FIGS. 11 to 13, FIGS. 19 to 21 and FIGS. 28 to 31, in another embodiment, the front panel 2 may comprise a first region 10 and a second region 12 which are configured to secure the front panel 2 to the abdomen area of a user when used in conjunction with back strap 4 having a first region 14 and second region 16 as shown in FIGS. 14, 15, 22, 26 and 27. In one embodiment, first region 10 and second region 12 may be located on the exterior side of the outer layer 8 as shown in FIGS. 11 to 13, 19, 20, 30 and 31. In another embodiment, first region 10 and second region 12 may be located on the exterior side of single layer 28 as shown in FIGS. 28 and 29. In one embodiment, first region 10 and second region 12 may be located at or near the hip region on opposite sides of front panel 2 and extend down to the pubic region as shown in FIGS. 11 to 13, 28 and 29. In a further embodiment, first region 10 and second region 12 may be located at or near the hip region on opposite sides of front panel 2 as shown in FIGS. 19, 20 and 30. The exact location of first region 10 and second region 12 may vary as desired by one of skill in the art.

First region 10 and second region 12 of front panel 2 are configured to function in conjunction with first region 14 and second region 16 of back strap 4 to form a closure structure which secures front panel 2 to the abdomen area of a user. In one embodiment, back strap 4 may be made of any semi stretch, smooth fabric, textile or blend of textiles such as cotton, nylon and/or elastic as desired by one of skill in the art. Any textile or blend of textiles may be used as long as such textile or blend of textiles is capable of stretching (such as four way stretching) and moisture wicking. In one embodiment, back strap 4 may comprise any textile or blended textile that is preshrunk.

Referring to FIGS. 14, 15, 22, 26 and 27, back strap 4 may be various configurations and dimensions as desired by one of skill in the art. The configuration and dimensions of back strap 4 having first region 14 and second region 16 may be dependent on the dimensions of first region 10 and second region 12 of front panel 2 with which back strap 4 is functioning to form a closure structure and secure the front panel 2 to a user. The closure structure may include hook and loop fasteners (such as Velcro), snap fasteners, button fasteners, lace fastener, hook fasteners and/or any mixtures thereof.

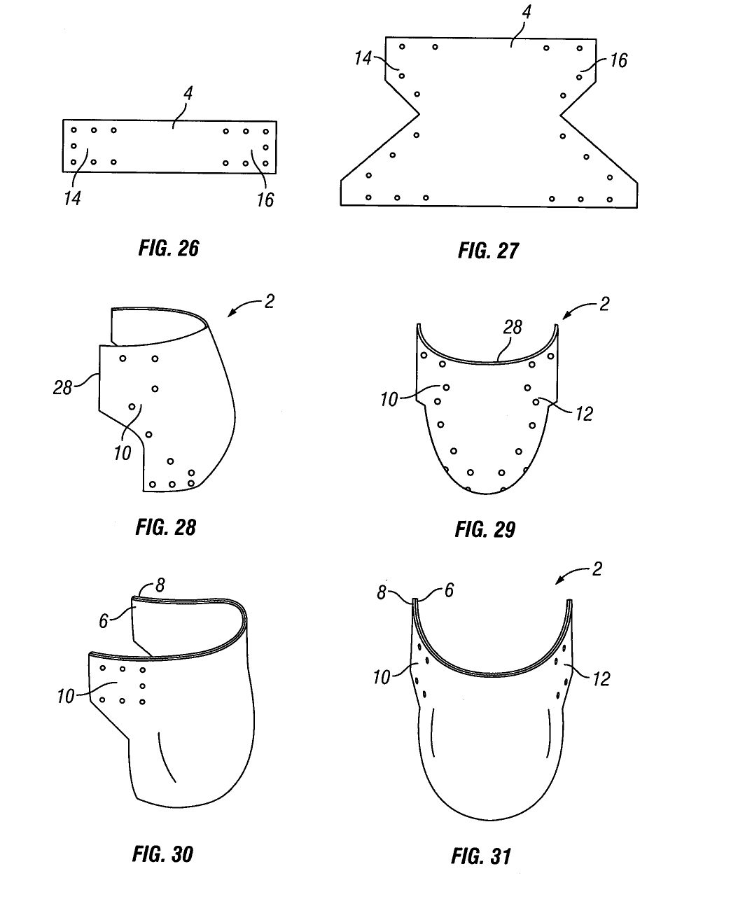

Referring now to FIGS. 16 to 18, in one example embodiment, front panel 2 as shown in FIGS. 11 to 13 is secured to the abdomen area of a user with back strap 4 as shown in FIGS. 14 to 15. First region 14 of back strap 4 secures to first region 10 of front panel 2. Second region 16 of back strap 4 secures to second region 12 of front panel 2. In such embodiment, first region 10 and second region 12 extend from the upper hip region to the lower pubic region along opposite sides of the front panel 2. First region 14 and second region 16 are configured to function with first region 10 and second region 12. In such embodiment, hook and loop fasteners may be used to secure the front panel 2 to the abdomen area with back strap 4 to form a closure structure. In another embodiment, snap fasteners and/or buttons may be used to form a closure structure as shown in FIGS. 26 to 29. Any other type or combination of fasteners may be used as desired by one skilled in the art.

Referring now to FIGS. 23 to 25, in another example embodiment, front panel 2 as shown in FIGS. 19 to 21 is secured to the abdomen area of a user with back strap 4 as shown in FIG. 22. First region 14 of back strap 4 secures to first region 10 of front panel 2. Second region 16 of back strap 4 secures to second region 12 of front panel 2. In such embodiment, first region 10 and second region 12 are located at the upper hip region on opposite sides of the front panel 2. First region 14 and second region 16 are configured to function with first region 10 and second region 12. In such embodiment, hook and loop fasteners may be used to secure the front panel 2 to the abdomen area with back strap 4 to form a closure structure. In another embodiment, snap fasteners and/or buttons may be used to form a closure structure as shown in FIGS. 30 and 31. Any other type or combination of fasteners may be used as desired by one skilled in the art.

A method for providing hydration and elasticity to the skin of the abdomen area is also disclosed. In one embodiment, the method may comprise the following steps: (a) obtaining a garment 1 comprising a detachable front panel 2 configured to encase and secure to the abdomen area, wherein the front panel 2 comprises an inner layer 6 having an interior side and an exterior side and an outer layer 8 having an interior side and an exterior side, wherein the exterior side of the inner layer 6 is contiguous to the interior side of the outer layer 8, wherein the inner layer 6 comprises a gelatinous material comprising proteins; (b) securing the front panel 2 to the abdomen area; and (c) wearing the front panel 2 for a period of time. In one embodiment, the method may further include the following steps: step (d) removing the front panel 2 and step (e) repeating steps (b) to (c) as needed.

In one embodiment, the interior side of the inner layer 6 may comprise a self-adhesive edge 26 which secures the front panel 2 to the abdomen area. In another embodiment, the garment 1 may comprise a back strap 4 which secures the front panel 2 to the abdomen area at a first region 10 and a second region 12.

In one embodiment, the gelatinous material may further comprise the following ingredients: hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter, and any mixtures thereof.

Another exemplary method for providing hydration and elasticity to the skin of the abdomen area is disclosed. The method may comprise the following steps: (a) obtaining a garment 1 comprising a detachable front panel 2 configured to encase and secure to the abdomen area, wherein the front panel 2 may comprise a single layer 28 having an interior side 30 and an exterior side 32, wherein the interior side is pretreated with micro particles and/or micro capsules comprising vitamins and ingredients having skin nourishing and skin regenerative qualities; (b) securing the front panel 2 to the abdomen area; and (c) wearing the front panel 2 for a period of time. In one embodiment, the method may further include the following steps: step (d) removing the front panel 2 and step (e) repeating steps (b) to (c) as needed.

In one embodiment, the interior side of the front panel 2 may comprise a self-adhesive edge 26 which secures the front panel 2 to the abdomen area. In another embodiment, the micro particles and/or micro capsules may comprise the following ingredients: proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and/or any mixtures thereof.

In one embodiment, the method may comprise wearing front panel 2 for an extended period of time such as throughout the night while the user sleeps. In another embodiment, the method may comprise wearing the garment for a shorter period of such as twenty minutes. The garment 1 may be worn for any period of time as desired by one of skill in the art. In one embodiment, the method may include repeating steps (b) and (c) as needed to hydrate the abdomen area. In one embodiment, the method may include repeating steps (b) and (c) each night for the duration of the pregnancy. In another embodiment, the method may include repeating steps (b) and (c) for any other length or duration of time as desired by one of skill in the art.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the disclosed invention and the equivalents thereof.

1. A garment used to provide hydration and elasticity to the skin of the abdomen area, the garment comprising:
a detachable front panel configured to encase and secure to the abdomen area, wherein the front panel comprises an inner layer having an interior side and an exterior side and an outer layer having an interior side and an exterior side, wherein the exterior side of the inner layer is contiguous to the interior side of the outer layer, wherein the inner layer comprises a gelatinous material comprising proteins.

2. The garment of claim 1 wherein the interior side of the inner layer comprises a self-adhesive edge which secures the front panel to the abdomen area.

3. The garment of claim 1 wherein the garment comprises a back strap which secures the front panel to the abdomen area at a first region and a second region.

4. The garment of claim 3 wherein the back strap is secured to the front panel by a closure structure selected from a group consisting of hook and loop fasteners, snap fasteners, button fasteners, lace fastener, hook fasteners and mixtures thereof.

5. The garment of claim 1 wherein the gelatinous material further comprises ingredients selected from the group consisting of hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and mixtures thereof.

6. A garment used to provide hydration and elasticity to the skin of the abdomen area, the garment comprising:
a detachable front panel configured to encase and secure to the abdomen area, wherein the front panel comprises a single layer having an interior side and an exterior side, wherein the interior side is pretreated with micro particles and/or micro capsules comprising vitamins and ingredients having skin nourishing and skin regenerative qualities.

7. The garment of claim 6 wherein the interior side of the front panel comprises a self-adhesive edge which secures the front panel to the abdomen area.

8. The garment of claim 6 wherein the garment comprises a back strap which secures the front panel to the abdomen area at a first and second region.

9. The garment of claim 8 wherein the back strap is secured to the front panel by a closure structure selected from the group consisting of hook and loop fasteners, snap fasteners, button fasteners, lace fastener, hook fasteners and mixtures thereof.

10. The garment of claim 6 wherein the micro particles and/or micro capsules comprise ingredients selected from the group consisting of proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and mixtures thereof.

11. A method for providing hydration and elasticity to the skin of the abdomen area, the method comprising the following steps:
(a) obtaining a garment comprising a detachable front panel configured to encase and secure to the abdomen area, wherein the front panel comprises an inner layer having an interior side and an exterior side and an outer layer having an interior side and an exterior side, wherein the exterior side of the inner layer is contiguous to the interior side of the outer layer, wherein the inner layer comprises a gelatinous material comprising proteins;
(b) securing the front panel to the abdomen area; and
(c) wearing the front panel for a period of time.

12. The method of claim 11 wherein the interior side of the inner layer comprises a self-adhesive edge which secures the front panel to the abdomen area.

13. The method of claim 11 wherein the garment comprises a back strap which secures the front panel to the abdomen area at a first region and a second region.

14. The method of claim 11 wherein the gelatinous material further comprises ingredients selected from the group consisting of hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter, and mixtures thereof.

15. The method of claim 11 wherein the method further includes step (d) removing the front panel and step (e) repeating steps (b) to (c) as needed.

16. A method for providing hydration and elasticity to the skin of the abdomen area, the method comprising the following steps:
(a) obtaining a garment comprising a detachable front panel configured to encase and secure to the abdomen area, wherein the front panel comprises a single layer having an interior side and an exterior side, wherein the interior side is pretreated with micro particles and/or micro capsules comprising vitamins and ingredients having skin nourishing and skin regenerative qualities;
(b) securing the front panel to the abdomen area; and
(c) wearing the front panel for a period of time.

17. The method of claim 16 wherein the interior side of the front panel comprises a self-adhesive edge which secures the front panel to the abdomen area.

18. The method of claim 16 wherein the micro particles and/or micro capsules comprise ingredients selected from the group consisting of proteins, hydrolyzed collagen, hydrolyzed elastin, hydrolyzed soy protein, vitamin E, vitamin D3, vitamin A, aloe vera, grapefruit seed extract, beeswax, sweet almond oil, argon oil, olive oil, squalene oil, jojoba oil, DL panthenol, shea butter, cocoa butter and mixtures thereof.

19. The method of claim 16 wherein the method further includes the steps (d) removing the front panel and step (e) repeating steps (b) to (c) as needed.
